(19) United States
(12) Patent Application Publication
(10) Pub. No.: US 2013/0059924 A1
(43) Pub. Date: Mar. 7, 2013

(54) PHOTOSTABILISERS

(71) Applicants: René Peter Scheurich, Gross-Zimmern (DE); Thomas Rudolph, Darmstadt (DE); Junyou Pan, Frankfurt am Main (DE)

(72) Inventors: René Peter Scheurich, Gross-Zimmern (DE); Thomas Rudolph, Darmstadt (DE); Junyou Pan, Frankfurt am Main (DE)

(73) Assignee: MERCK PATENT GESELLSCHAFT MIT BESCHRANKTER HAFTUNG, DARMSTADT (DE)

(21) Appl. No.: 13/697,416

(22) PCT Filed: Apr. 13, 2011
(86) PCT No.: PCT/EP2011/001871

§ 371 Date: Nov. 12, 2012

(30) Foreign Application Priority Data
May 12, 2010 (EP) 10005019.4

(51) Int. Cl. A61K 47/08; A23L 1/00; G02B 5/20; C07C 49/84; A61K 8/35

(52) U.S. Cl. 514/772; 568/331; 252/589; 426/531

(57) ABSTRACT

The present invention relates to the use of specific aldehydes or ketones of the formula I as photostabiliser for at least one compound to be stabilised. The invention furthermore relates to a method for the photostabilisation of a second compound by a compound of the formula I and to compositions comprising at least one compound of the formula I of this type.

The present invention relates to the use of a compound of the formula I as photostabiliser for at least one compound to be stabilised. The invention furthermore relates to a method for the photostabilisation of a second compound by a compound of the formula I and to compositions comprising at least one compound of the formula I of this type.

Solar radiation or also radiation from artificial sources can interact with photoactive ingredients of materials. Photoactive ingredients of this type can be compounds, such as, for example, pigments, UV filters, polymers, antioxidants, plastics, vitamins, fragrances or other photoactive compounds. Owing to the absorption of radiation by photoactive ingredients of this type, however, the latter may experience a structural change due to a photochemical reaction, which can result in impairment of the product properties or decomposition. This photochemical degradation of photoactive ingredients of this type can result, for example in the case of dyes, in fading or a loss of gloss, in the case of cosmetic products in discoloration or the formation of unpleasant odours. In the case of packaging materials, the respective packaging material may become brittle and lose its protective function. Photodegradation of this type can proceed by a very wide variety of reaction mechanisms and in a differing number of steps. In at least one step, a photoactive ingredient of this type will absorb the radiation, where the respective photoactive compound can be converted into an excited electronic state by the absorbed radiation energy. A photoactive compound of this type can in turn transfer from this excited state into further excited electronic states of the same or different spin multiplicity (inter system crossing) or be returned to the ground state (relaxation). If relaxation of this type to the electronic ground state does not occur sufficiently quickly and if the respective photoactive compound is also unstable in at least one of the excited higher energy levels owing to the higher energy content of the molecule, the molecule of the respective photoactive compound may experience a photochemical reaction, which may cause it to change with respect to its chemical structure.

One possibility for the photostabilisation of photoactive compounds of this type is the use of UV filters. The use of UV filters in compositions which comprise photoactive compounds or ingredients at least reduces the amount of radiation to which the photoactive compounds are exposed, so that the photoactive compounds are thus excited to a higher energy level less frequently and are thus converted by a photochemical reaction to a reduced extent. The use of UV filters thus enables a composition comprising photoactive compounds to be at least partially protected against the destructive UV radiation. The UV filters here are likewise photoactive compounds, which may likewise tend towards photodegradation.

EP 1 406 582 discloses UV filters which additionally have antioxidant properties. In accordance with the invention, compounds such as, for example, cinnamic acid derivatives and phenylethenyl malonates are used as UV filters of this type having antioxidant properties.

WO 2005/082325 describes the photostabilisation of sun-protection filter compositions, where the photostabilisation is achieved by admixtures of α-cyano-β,β-diphenyl acrylate compounds, such as, for example, octocrylenes, and a diester or polyester of a dicarboxynaphthalene. This photostabilisation takes place, for example in the case of a dibenzoylmethane, through a triplet-triplet energy transfer from a dibenzoylmethane of this type to a diester or polyester of a dicarboxynaphthalene, so that the dibenzoylmethane located in the triplet level falls back into the ground state, while one of the photostabilising compounds absorbs the triplet energy of the dibenzoylmethane and is itself raised into a triplet state.

Photostabilisation is achieved in accordance with U.S. Pat. No. 7,357,919 B2 by admixtures of arylalkyl benzoates and at least one compound which is able to absorb the triplet energy of an excited dibenzoylmethane by triplet-triplet energy transfer. The compounds which are able to absorb the triplet energy of the excited dibenzoylmethane have an energy level of a first excited triplet state of 1.73 to 3.03 eV.

US 2008/0131381 A1 discloses a method for the photostabilisation of photosensitive ingredients in compositions, such as cosmetics and household products, where the photostabilisation is carried out by dialkylbenzal malonates and UVA and UVB filters. Through combination of these three substance classes, adequate protection of the photosensitive ingredients against UV radiation is achieved.

The present invention relates to the problem of providing an alternative solution for the photostabilisation of photoactive compounds.

This problem is solved in accordance with the invention by the subject-matters of the independent claims. Advantageous embodiments are subject-matters of the dependent claims.

Surprisingly, it has been found that compounds of the formula I are photostabilisers, in particular for photoactive, photosensitive or photounstable compounds.

Hereinbelow, for example, the following definitions apply, in each case independently of one another:

A C₁-C₄-alkyl group stands for methyl, ethyl, propyl, isopropyl, butyl, 1-methylpropyl, 2-methylpropyl or tert-butyl.

A C₁-C₈-alkyl group stands for the substituents of the C₁-C₄-alkyl group or pentyl, hexyl, heptyl, octyl, 1-methylbutyl, 2-methylbutyl, 3-methylbutyl, 1-methylpentyl, 2-methylpentyl, 3-methylpentyl, 4-methylpentyl, 1-methylhexyl, 2-methylhexyl, 3-methylhexyl, 4-methylhexyl, 5-methylhexyl, 1-ethylpentyl, 2-ethylpentyl, 3-ethylpentyl, 1-ethylhexyl, 2-ethylhexyl, 3-ethylhexyl, 4-ethylhexyl.

A C₁-C₂₀-alkyl group stands for the substituents of the C₁-C₈-alkyl group or nonanyl, decanyl, undecanyl, dodecanyl, tridecanyl, tetradecanyl, pentadecanyl, hexadecanyl, heptadecanyl, octadecanyl, nonadecanyl, eicosanyl.

A C₂-C₄-alkenyl group stands for ethenyl, prop-1-enyl-, prop-2-enyl-isopropenyl, but-1-enyl, but-2-enyl, butadienyl, but-3-enyl.

A C₂-C₈-alkenyl group stands for the substituents of the C₁-C₄-alkenyl group or pentenyl, hexenyl, heptenyl, octenyl.

A C₂-C₂₀-alkenyl group stands for the substituents of the C₁-C₈-alkenyl group or octadec-9-enyl, octadec-9,12-dienyl, octadec-9,12,15-trienyl, nonadec-10-enyl, nonadec-10,13-dienyl, nonadec-10,13,16-trienyl, eicos-11-enyl, eicos-11,14-dienyl, eicos-11,14,17-trienyl.

A C₅-C₆-cycloalkyl group stands for cyclopentyl or cyclohexyl.

A C₃-C₈-cycloalkyl group stands for the substituents of the C₅-C₆-cycloalkyl group or cyclopropyl, cyclobutanyl, cycloheptyl or cyclooctyl, methylcylclohexyl, ethylcyclohexyl, 2,3-dimethylcyclohexyl, 2,4-dimethylcyclohexyl.

A C₃-C₈-cycloalkenyl group having at least one double bond stands for cyclopropenyl, cyclobutenyl, cyclopentyl, cyclopentadienyl, cyclohexenyl, cyclohexadienyl, cyclooctadienyl.

The invention thus relates to the use, in particular the non-therapeutic use, of a compound of the formula I

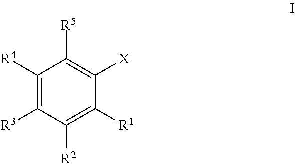

where X stands for

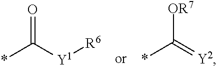

where the radical Y¹ stands for a single bond or the radicals Y¹ and Y² stand for a CR⁵R⁹ group,
where the radicals R¹ to R⁵ each, independently of one another, stand for H, Hal, CN, OH, C(═O)R¹⁰, NH₂, R¹¹ or OR¹¹,
where the radicals R⁶, R⁸ and R⁹ each, independently of one another, stand for H or R¹¹,
where the radical R⁷ stands for H, Si(R¹²)₃ or R¹¹,
where the radical R¹⁹ stands for OH, Hal, NH₂ or OR¹¹,
where the radicals R¹¹ each, independently of one another, stand for a straight-chain or branched C₁- to C₂₀-alkyl group or a straight-chain or branched C₂- to C₂₀-alkenyl group having at least one double bond, which may contain at least one OH bonded to a primary or secondary C atom, or for a C₃- to C₈-cycloalkyl group or for a C₃- to C₈-cycloalkenyl group having at least one double bond,
where the radicals R¹² each, independently of one another, stand for a straight-chain or branched C₁- to C₈-alkyl group,
where Hal stands for F, CI, Br or I,
and/or salts thereof as photostabiliser.

A further central idea of the invention is to select the energy levels of the first triplet state of the compound of the formula I and of a compound to be stabilised by the compound of the formula I in each case in pairs so that the energy levels of the first triplet states of the respective compound pairs differ from one another by a maximum of +/−5%.

A compound of this type to be stabilised is a photoactive, photosensitive or photounstable compound, which may optionally at least in some cases tend towards decomposition on excitation by, for example, UV rays or sunlight. Likewise, a stabilising compound can be taken to mean a photoactive, photosensitive or photounstable plastic.

The energy level of the first triplet state of the compound of the formula I and/or of the compound to be stabilised is preferably arranged in the range from 2.8 to 3.2 eV.

The energy level of the first triplet state of the compound of the formula I and/or of the compound to be stabilised is particularly preferably arranged in the range from 2.9 to 3.2 eV.

Irradiation of molecules of the compound(s) to be stabilised with UV rays enables them to absorb the radiation energy. The molecules here are usually raised from a ground state S₀ into an excited first singlet state S₁ or a higher singlet state S. The molecules can change from a singlet state of this type into a triplet state by intersystem crossing (ISC), and fall back from there into the ground state by, for example, thermal relaxation or radiation relaxation.

It is now possible that compounds to be stabilised, preferably in the first excited triplet state T₁, have a tendency towards decomposition. This is the case, in particular, if the triplet state is long-lived and there is no possibility to provide assistance by rapid triplet-triplet transfer with a suitable quench molecule.

A corresponding compound to be stabilised may decompose merely owing to the energy content of the first excited triplet state T₁ or owing to absorption of further energy by means of the UV radiation. An example which may be mentioned of a photochemical reaction of this type is the Norrish type I reaction of photochemical cleavage of aldehydes and ketones.

If the first excited triplet state of the respective compound is particularly susceptible to a spontaneous chemical decomposition reaction, it is advisable to leave precisely this first excited triplet state T₁ as quickly as possible in order that the statistical probability of decomposition of the compound becomes as low as possible. This can be achieved using a compound of the formula I as photostabiliser, where precisely this compound of the formula I is able to absorb the triplet energy of the compound to be stabilised which tends towards photochemical decomposition. Through a triplet-triplet energy transfer of this type between the first and second compound, the second compound is relaxed from its first excited triplet state T₁ into its ground state S₀, while the first compound absorbs the triplet energy being released in the process and is thus raised into its first triplet state T₁.

A triplet-triplet energy transfer of this type can proceed by the Förster mechanism or by the Dexter mechanism, where the triplet-triplet energy transfer in accordance with the Förster mechanism takes place through dipole-dipole interaction, while the triplet-triplet energy transfer by the Dexter mechanism takes place through electron exchange between the respective molecules on collision thereof.

The energy level of the first excited triplet state T₁ is usually about 0.5 eV below the energy level of the first excited singlet state S₁. However, compounds, such as, for example, avobenzone, are also known in which the energy level of the first excited triplet state T₁ is less than 0.5 eV below the energy level of the first excited singlet state S₁. In these cases, intersystem crossing (ISC), i.e. a transition of the molecule from the first excited singlet state S₁ to the first excited triplet state T₁, is particularly simple. A triplet state usually has a non-free-radical character.

A triplet-triplet energy transfer between in each case one molecule of the compound of the formula I and the compound to be stabilised takes place more simply the closer the two T₁ energy levels of the two molecules interacting with one another are to one another. Thus, a compound of the formula I is preferably employed for the photostabilisation of a compound to be stabilised if the energy levels of the first two triplet states T₁ are a maximum of +/−5% from one another.

Thus, a triplet-triplet energy transfer is preferably carried out if the triplet states T₁ of the compound of the formula I and of the compound to be stabilised are a maximum of +/−5% from one another.

Organic compounds can be described by the limiting orbitals HOMO (highest occupied molecular orbital; also known as conduction band) and LUMO (lowest unoccupied molecule orbital; also known as valence band) and by the band gap (the value of the energy difference between HOMO and LUMO). The energies can be determined experimentally here, for example, by means of CV (cyclic voltammetry), XPS (X-ray photoelectron spectroscopy) or UPS (ultraviolet photoelectron spectroscopy). The values can also be calculated by means of quantum-mechanical methods, for example by means of time-dependent density functional theory (DFT).

The energy levels of the respective singlet or triplet states of the respective first or second compound are preferably determined using a calibrated calculation method.

In the calibrated calculation method, quantum simulations regarding the energy levels are carried out in a first step by means of the Gaussian 03W software from Gaussian Inc. To this end, the molecular geometry is optimised by means of the semi-empirical quantum-chemical method AM1 (“Austin Model 1”), and the TD-DFT (time dependent density function theory) is then applied, where the energy calculations for the HOMO/LUMO energy levels and the energy levels for the excited triplet and singlet states are carried out by means of the density functional theory method B3PW91 and the function base set 6-31G(d).

In addition, the calculated HOMO and LUMO energy levels are corrected in a second step by means of cyclic voltammetry. For this purpose, selected compounds are measured by cyclic voltammetry and in parallel calculated using the Gaussian 03W software by the method described above including the density functional theory method B3PW91 using the same function base set 6-31G(d). The calculated values for these selected compounds are compared with the values measured by cyclic voltammetry, and calibration factors are determined from the differences. These calibration factors are used for future calculations of compounds which have similar structures, to the selected compounds.

In order to verify this calibrated calculation method, the T₁ energy levels were likewise measured by means of time-resolved spectroscopy at lower temperatures. To this end, an approximately 100 nm thick film, of the organic compound of interest is embedded in quartz and then excited using a YAG laser or an N₂ laser at 10 kelvin, and the photoluminescence spectrum emitted is recorded after 10 μs. The T₁ energy levels were then determined from the photoluminescence spectrum recorded. It was observed here that the agreement between the T₁ energy levels calculated by the above method and the measured T₁ energy levels with respect to the selected compounds is accurate to two decimal points.

In the case of the enol forms of the compound of the formula I where

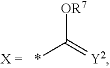

the radical R⁷ preferably stands for Si(R¹²)₃ and R¹² has in each case independently a meaning as described above. All three radicals R¹² are preferably identical and are selected from a straight-chain or branched alkyl group having 1 to 4 C atoms.

The radical R⁷ particularly preferably stands for a trimethylsilyl group (TMS) or a tri-tert-butylsilyl group (TBDMS).

The radicals R¹¹ in formula I preferably each stand, independently of one another, for a straight-chain or branched C₁- to C₈-alkyl group or a straight-chain or branched C₁- to C₈-alkenyl group having at least one double bond, which may contain at least one OH bonded to a primary or secondary C atom.

The radicals R¹¹ particularly preferably stand, in each case independently of one another, for a straight-chain or branched C₁- to C₈-alkyl group, very particularly preferably for a straight-chain or branched C₁- to C₄-alkyl group.

The radicals R¹¹ may also stand for a C₅ or C₆-cycloalkyl group.

The radicals Y¹ and Y² in formula I preferably stand for CR⁸R⁹, where R⁸ and R⁹ each independently stand for H or R¹¹, where R¹¹ has one of the meanings indicated above or meanings indicated as preferred.

Alternatively, Y¹ in formula I preferably stands for a single bond and R⁶ stands for H.

The radicals R⁶ and R⁸ in the compounds of the formula I preferably stand for H.

The radical R⁹ preferably stands for R¹¹ or H, where R¹¹ has one of the meanings indicated above or meanings indicated as preferred.

Acetophenones, propiophenones or benzaldehydes of the formula I are preferably used in accordance with the invention.

In the case of acetophenones of the formula I, X stands for

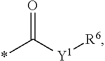

Y¹ stands for CR⁸R⁹, R⁸ stands for H, R⁹ stands for H and R⁶ stands for H.

In the case of propiophenones of the formula I, X stands for

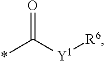

Y¹ stands for CR⁸R⁹, R⁸ stands for H, R⁹ stands for methyl and R⁶ stands for H.

In the case of benzaldehydes of the formula I, X stands for

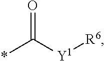

Y¹ stands for a single bond and R⁶ stands for H.

In the case of the radicals R¹ to R⁵ of the benzene ring, it is preferred for at least two of the radicals to stand for an H atom.

The radicals R¹ to R⁵ are each, independently of one another, preferably H, CH₃, C₂H₅, CH(CH₃)₂, C(CH₃)₃, OCH₃, OC₂H₅, CF₃, CH═CH₂, CH₂OH, (C═O)OH, (C═O)Cl, (C═O)OCH₃, (C═O)OC₂H₅, (C═O)NH₂ or CN.

It is furthermore preferred for the radical R³ to denote R¹¹ or OR¹¹, where R¹¹ has one of the meanings indicated above or meanings indicated as preferred.

The radicals R¹, R², R⁴, R⁵ are likewise preferably H. In particular, the radical R³ denotes OCH₃, OC₂H₅, CH(CH₃)₂ or C(CH₃)₂.

Particular preference is given to acetophenones or propiophenones of the formula I, as described above, in which the radicals R¹, R², R⁴ and R⁵ stand for an H atom.

Particular preference is likewise given to acetophenones or propiophenones of the formula I, as described above, in which the radical R³ stands for OCH₃, OC₂H₅, CH(CH₃)₂ or C(CH₃)₂.

Since ketones, in accordance with keto-enol tautomerism, usually may also exist in their isomeric enol form to a pre-determined proportion besides their keto form and these two forms interconvert owing to keto-enol tautomerism from a pre-determined limiting temperature, all enol forms of the ketones of the formula I are thus explicitly also suitable and claimed for use as photostabiliser for a compound to be stabilised.

Particularly preferred benzaldehydes, as described above, are benzaldehydes of the formula I in which the radicals R¹, R², R⁴ and R⁵ stand for H.

Particular preference is likewise given to benzaldehydes, as described above, in which the radical R³ stands for OCH₃, OC₂H₅, CH(CH₃)₂ or C(CH₃)₂.

In particular, the following compounds of the formula I are preferred as individual compounds for the photostabilisation of a compound to be stabilised:

4-methoxyacetophenone; 3-methoxyacetophenone, 2-methoxyacetophenone, acetophenone, 3,4-dimethoxyacetophenone, 4-tert-butylacetophenone, 3-tert-butylacetophenone 4-methylacetophenone, 3-methylacetophenone, 2-methylacetophenone, 4-isopropylacetophenone, 3-isopropylacetophenone, 4-hydroxyacetophenone, 3-hydroxyacetophenone, 2-hydroxyacetophenone, 6-amino-2-hydroxyacetophenone, 2,4-dimethylacetophenone, 2,3-dihydroxyacetophenone, 2,4-dihydroxyacetophenone, 2,5-dihydroxaacetophenone, 2-aminoacetophenone, 3-aminoacetophenone, 4-aminoacetophenone, 4-methoxybenzaldehyde, 3-methoxybenzaldehyde, 2-methoxybenzaldehyde, 3,4-dimethoxybenzaldehyde, 3,5-dimethoxybenzaldehyde, 3,4,5-trimethoxybenzaldehyde, 4-isopropylbenzaldehyde, 3-isopropylbenzaldehyde, 4-tert-butylbenzaldehyde, 3-tert-butylbenzaldehyde; benzaldehyde, 2-methylbenzaldehyde, 3-methylbenzaldehyde, 4-methylbenzaldehyde, 2-hydroxybenzaldehyde, 3-hydroxybenzaldehyde, 4-hydroxybenzaldehyde, 2-aminobenzaldehyde, 3-aminobenzaldehyde, 4-aminobenzaldehyde, 2,6-dimethylbenzaldehyde, 2,4-dimethylbenzaldehyde, 4-ethylbenzaldehyde, 2,5-dimethylbenzaldehyde, 3,4-di methylbenzaldehyde.

A compound to be stabilised which is intended to be photostabilised by a compound of the formula I is distinguished by photoinstability, in particular of a first triplet state of one of its appearance forms.

A compound to be stabilised here can also be taken to mean a plastic which tends towards photoinstability and has properties described above and below of the compound to be stabilised.

A compound of this type to be stabilised preferably has an energy level of the first triplet state T₁, calculated by the calibrated calculation method, which is between 2.8 eV and 3.2 eV or particularly preferably between 2.9 eV and 3.2 eV. The compound to be stabilised is energetically relaxed and returned to the ground state Sₒ by triplet-triplet energy transfer from the compound to be stabilised to the compound of the formula I or to the embodiments described as preferred above, enabling photochemical decomposition of the compound to be stabilised to be at least reduced.

An appearance form of the compound to be stabilised which tends towards photochemical decomposition can be taken to mean, for example, an isomeric structure of the respective compound to be stabilised, where the isomeric structures or appearance forms of this compound to be stabilised usually exist alongside one another in a pre-determined ratio to one another and can easily interconvert at a pre-determined temperature, as is the case, for example, in the case of keto-enol tautomers. Possible appearance forms are thus, for example, the tautomers of keto-enol tautomerism, ketol-enediol tautomerism, amide-imidic acid tautomerism, imine-enamine tautomerism, lactam-lactim tautomerism, thiolactam-thiolactim tautomerism, oxy-cyclo tautomerism, cyclopropyl-homoallyl tautomerism or tropanol-cycloheptanone tautomerism. However, protonated or deprotonated compounds may also represent an appearance form of this type, such as, for example, enolates or protonated amines.

Thus, compounds to be stabilised by compounds of the formula I, such as, for example, plastics, UV filters, antioxidants, vitamins, fragrances, surface coatings, self-tanning agents or the like can be photostabilised. The photounstable compounds to be stabilised are advantageously thereby protected against harmful, decomposing radiation, where the compound of the formula I optionally decomposes owing to the transferred triplet energy, and transfer of the triplet energy back to the compound to be stabilised is thus suppressed.

Organic UV filters are preferably stabilised, in particular comprising the structural units dibenzoylmethane or triazine.

Dibenzoylmethane derivatives are products which are already well known per se and are described, in particular, in the above-mentioned specifications FR-A-2 326 405, FR-A-2 440 933 and EP-A-0 114 607. Preferred dibenzoylmethane derivatives conform to the formula C

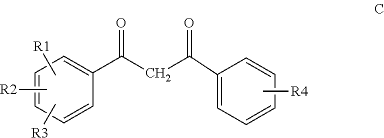

in which R1, R2, R3 and R4, which are identical or different from one another, denote hydrogen, a straight-chain or branched C₁₋₈-alkyl group or a straight-chain or branched C₁₋₈-alkoxy group, in particular
2-methyldibenzoylmethane,
4-methyldibenzoylmethane,
4-isopropyldigenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-methoxy-tert-butyldibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane and
2,6-dimethyl-4-tert-butyl-4′-methoxydibenzoylmethane, where this list is not limiting.

Of the above-mentioned dibenzoylmethane derivatives, particular preference is given in accordance with the invention to 4,4′-methoxy-tert-butyldibenzoylmethane and in particular 4,4′-methoxy-tert-butyldibenzoylmethane (BMDBM or avobenzone), which is commercially available under the trade name Eusolex® 9020 from Merck, where this filter conforms to the following structural formula:

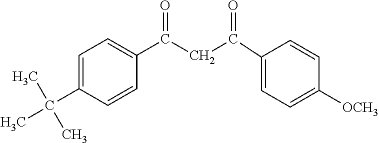 [SMILES: COc1ccc(C(=O)CC(=O)c2ccc(C(C)(C)C)cc2)cc1]

A further preferred dibenzoylmethane derivative is 4-isopropyldibenzoylmethane.

Preferred triazine derivatives conform to the formula I

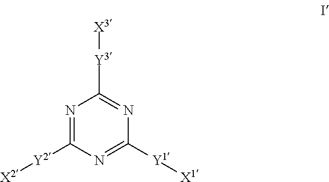

where Y^{1′}, Y^{2′} and Y^{3′} each stand, independently of one another, for a single bond or NH,
where X^{1′}, X^{2′} and X^{3′} each stand, independently of one another, for Alk¹ or a substituent of the formula II or III

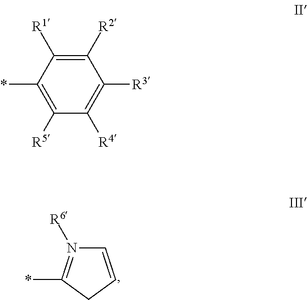

where R^{1′} to R^{5′} each stand, independently of one another, for H, OH, Hal, Alk¹, OAlk¹, SAlk¹, NHAlk¹, N(Alk¹)₂, COOAlk¹, COOH, C(O)H, CONHAlk¹, CONH₂, COO⁻Kt⁺, Cyc¹, OCyc¹, Arl¹, OArl¹, COOArl¹, biphenylyl, Het¹, OHet¹, Si(Alk²)₃, OEth, COOEth or for a substituent of the formula IV

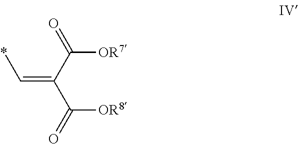

where Alk¹ in each case stands, independently of one another, for a straight-chain or branched C₁- to C₂₀-alkyl group or for a straight-chain or branched C₂- to C₂₀-alkenyl group which has at least one double bond or for a straight-chain or branched C₂- to C₂₀-alkynyl group which has at least one triple bond, and/or in which at least one or more non-adjacent C atoms of the alkyl, alkenyl or alkynyl group may be replaced by 0 or trimethylsilyl and/or which may contain at least one OH bonded to a primary or secondary C atom,
where Cyc¹ in each case stands, independently of one another, for a C₃- to C₈-cycloalkyl group, which may have at least one double bond, and/or in which at least one CH₂ may be replaced by O or NH,
where Arl¹ in each case stands, independently of one another, for an unsubstituted, mono- or polysubstituted C₆- to C₂₀-aryl group,
where Het¹ in each case stands, independently of one another, for an unsubstituted, mono- or polysubstituted C₅- to C₂₀-aryl group, in which at least one CH₂ has been replaced by O, S or NH,
where Alk² in each case stands, independently of one another,
for a straight-chain or branched C₁- to C₁₂-alkyl group, which may be interrupted by at least one O,
where R^{6′} stands for Alk¹,
where R^{7′} and R^{8′} each stand, independently of one another, for Alk¹ or Arl¹,
where Eth¹ in each case stands, independently of one another,
for —(CH₂—CH₂—O)ₘ₁-Alk², where 1≦m1≦16,
where Hal stands for F, Cl, Br or I,
where Kt⁺ stands for Li⁺, Na⁺ or K⁺,

Preferred individual compounds of the triazine derivatives of the formula I′ are compounds of the formulae X′, XI′, XII′, XIII′, XIV′, XV′, XVI′, XVII′, XVIII′, XIX′, XX′, XXI′ or XXII′

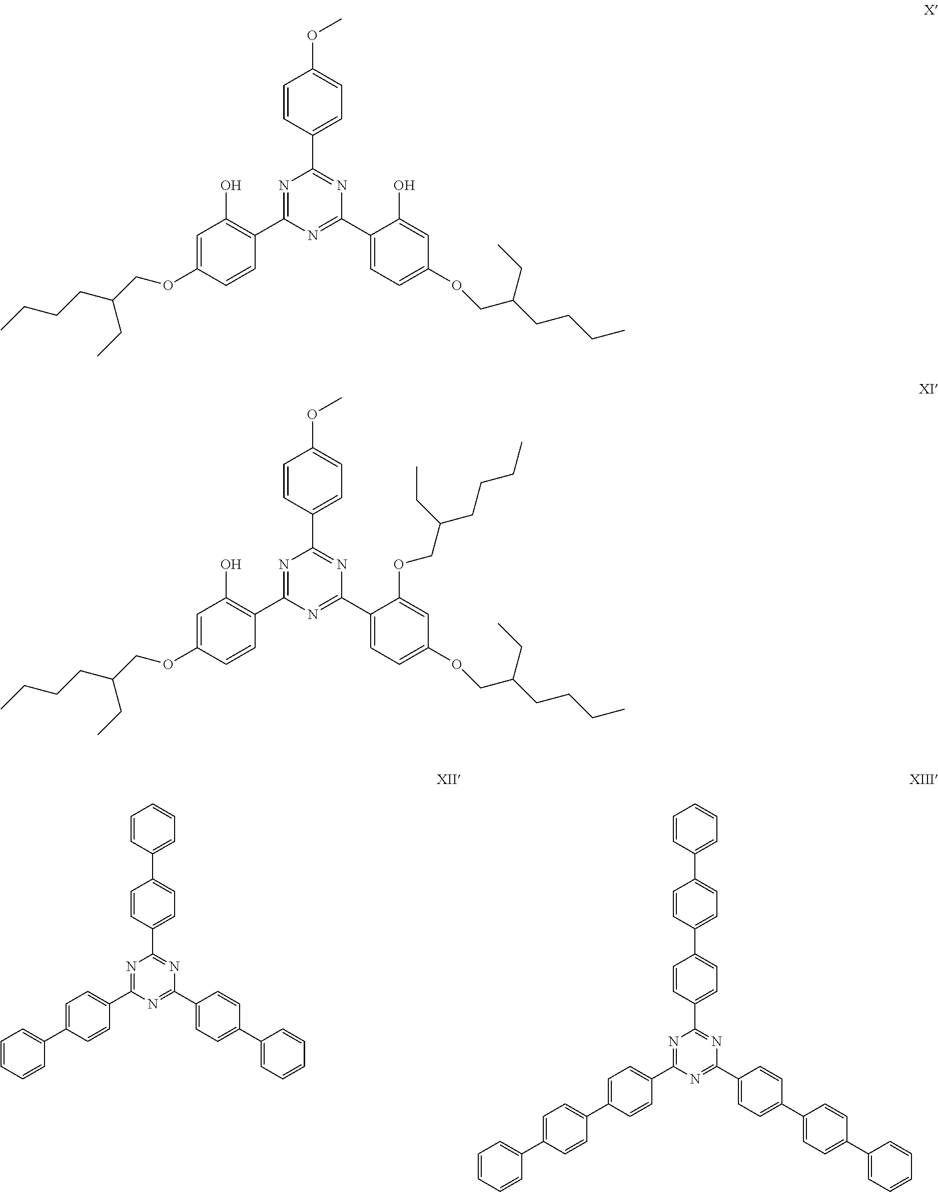

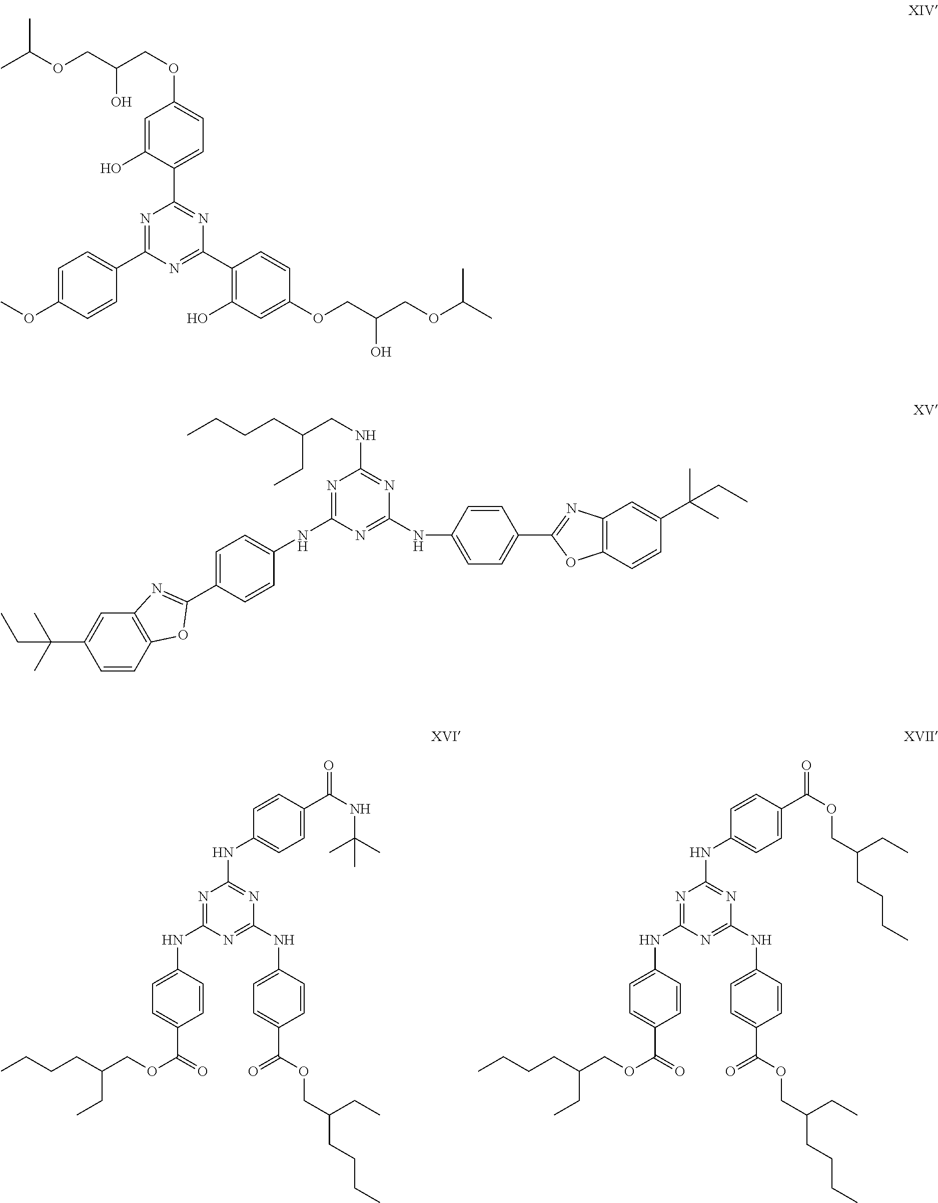

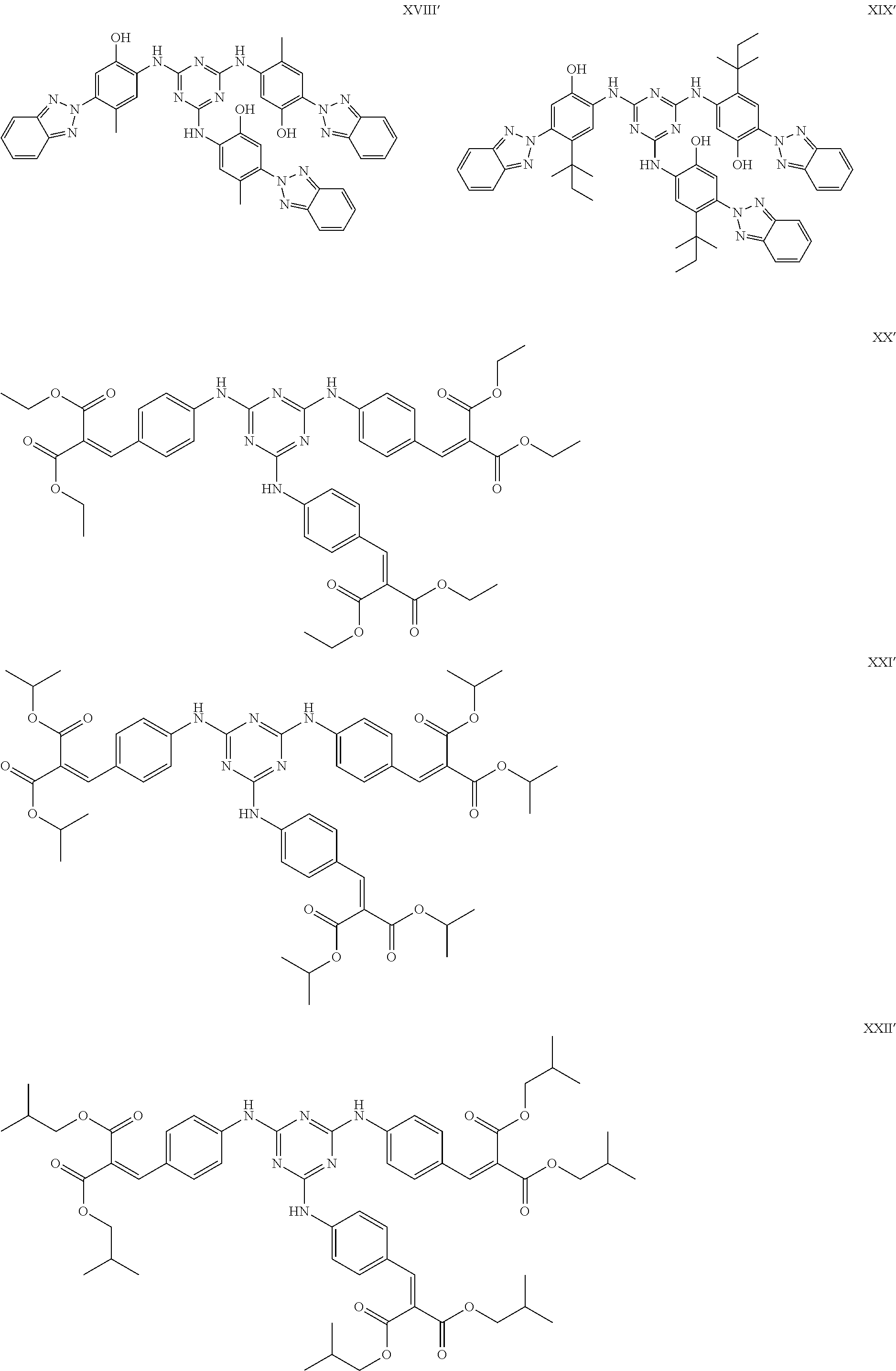

From this group of the individual compounds of the triazine derivatives, the individual compounds of the formula X, XII, XVI, XVII and XV, in particular, are photostabilised. Formula XVI corresponds to the UV filter Uvasorb® Heb from Sigma. Formula X corresponds to the UV filter Tinosorb® S. Formula XVII corresponds to the UV filter Uvinul T 150 from BASF.

The UV filters Uvasorb Heb, Tinosorb S, Uvinul T 150 or avobenzone are particularly preferably photostabilised. Azobenzone is very particularly preferably stabilised. The UV filters, in particular avobenzone, are very particularly preferably stabilised using the compound 4-methoxyacetophenone.

In the case of the photostabilization of avobenzone by 4-methoxyacetophenone, an advantageous compound pair is present, since the first triplet energy level of avobenzone at 3.1 eV is only 1.6% lower than the first triplet energy level of 4-methoxyacetophenone at 3.15 eV.

A further general idea of the invention is a method for the photostabilization of a compound to be stabilized by a first compound, in particular of the formula I, or one of the compounds of the formula I described as preferred, where the compound to be stabilized, excited to a first excited triplet state by radiation, in particular from a wavelength range of the wavelength 280 nm to 400 nm, may tend towards decomposition in this state, where the compound to be stabilized is stabilized by a triplet-triplet energy transfer between the compound of the formula I and the compound to be stabilized, which is in the first excited triplet state T₁.

The present invention furthermore relates to compositions comprising at least one vehicle which is suitable for cosmetic, pharmaceutical compositions, foods, food supplements, household products or packaging materials and at least one compound of the formula I or one of the compounds indicated as preferred.

The invention furthermore relates to a composition which comprise at least one compound to be stabilized together with at least one of the compounds of the formula I described above or indicated as preferred or the individual compounds listed. It is also possible here for a plurality of compounds to be stabilized to be photostabilized by one or more compounds of the formula I.

The compositions here are usually compositions which can be applied topically, for example cosmetic or dermatological formulations or medical devices. In this case, the compositions comprise a cosmetically or dermatologically suitable vehicle and, depending on the desired property profile, optionally further suitable ingredients. In the case of pharmaceutical compositions, the compositions in this case comprise a pharmaceutically tolerated vehicle and optionally further pharmaceutical active compounds.

“Can be applied topically” in the sense of the invention means that the composition is applied externally and locally, i.e. that the composition must be suitable for, for example, application to the skin.

Preferred compositions are cosmetic compositions.

In the sense of the present invention, the term agent or formulation is also used synonymously alongside the term composition.

The compositions may include or comprise, essentially consist of or consist of the said requisite or optional constituents. All compounds or components which can be used in the compositions are either known and commercially available or can be synthesised by known processes.

Further preferred combinations of embodiments are disclosed in the Claims.

The invention also relates to a process for the preparation of a composition, as described above, in which at least one compound of the formula I is mixed with a vehicle and optionally with further active compounds or assistants. Suitable vehicles and active compounds or assistants are described in detail in the following part.

In preferred embodiments, at least one compound of the formula I having the substituents which are defined or indicated as preferred or preferred individual compounds is typically employed in the compositions according to the invention in amounts of 0.05 to 20% by weight, preferably in amounts of 0.1% by weight to 20% by weight and particularly preferably in amounts of 0.5 to 20% by weight. The person skilled in the art is presented with absolutely no difficulties here in selecting the amounts correspondingly depending on the intended action of the composition.

In the compositions described which, in accordance with the invention, comprise at least one compound of the formula I, coloured pigments may furthermore also be present, where the layer structure of the pigments is not limited.

The coloured pigment should preferably be skin-coloured or brownish on use of 0.1 to 5% by weight. The choice of a corresponding pigment is familiar to the person skilled in the art.

Besides the compounds of the formula I and the second compounds to be stabilised and any other ingredients, preferred compositions comprise further organic UV filters, so-called hydrophilic or lipophilic sun-protection filters, which are effective in the UVA region and/or UVB region and/or IR and/or VIS region (absorbers). These substances can be selected, in particular, from cinnamic acid derivatives, salicylic acid derivatives, camphor derivatives, β,β-diphenylacrylate derivatives, p-aminobenzoic acid derivatives and polymeric filters and silicone filters, which are described in the application WO-93/04665. Further examples of organic filters are indicated in the patent application EP-A 0 487 404. The said UV filters are usually named below in accordance with INCI nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Haarmann and Reimer under the name “Neo Heliopan OS”, Dipropylene glycol salicylate, for example marketed by Scher under the name “Dipsal”, TEA salicylate, for example marketed by Haarmann and Reimer under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidenedicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Haarmann and Reimer under the name “Neo Heliopan AP”.

Phenylbenzotriazole derivatives: Drometrizole trisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by Ciba Specialty Chemicals under the name “Tinosorb M”.

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl)imino]-6-(2-ethylhexyl)imino-1,3,5-triazine, for example marketed by Sigma 3V under the name Uvasorb K2A, and mixtures comprising this.

piperazine derivatives, such as, for example, the compound

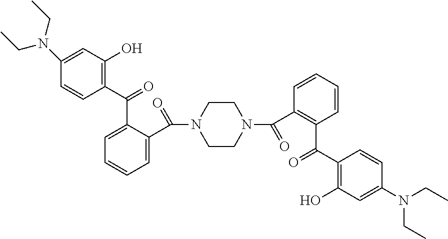 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

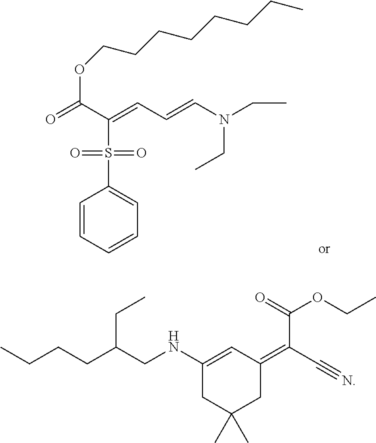

The compounds listed should only be regarded as examples. It is of course also possible to use other UV filters.

Suitable organic UV-protecting substances can preferably be selected from the following list: Ethylhexyl salicylate, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidenedicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, 2,4-Bis[5-1 dimethylpropyl)benzoxazol-2-yl(4-phenyl)imino]-6-(2-ethylhexyl)imino-1,3,5-triazine and mixtures thereof.

These organic UV filters are generally incorporated into formulations in an amount of 0.01 percent by weight to 20 percent by weight, preferably 1% by weight-10% by weight.

Besides the compounds of the formula I and any other organic UV filters, as described above, preferred compositions comprise further inorganic UV filters, so-called particulate UV filters.

These combinations with particulate UV filters are possible both as powder and also as dispersion or paste of the following types.

Preference is given here both to those from the group of the titanium dioxides, such as, for example, coated titanium dioxide (for example Eusolex® T-2000, Eusolex®T-AQUA, Eusolex®T-AVO, Eusolex®T-OLEO), zinc oxides (for example Sachtotec), iron oxides or also cerium oxides and/or zirconium oxides.

Furthermore, combinations with pigmentary titanium dioxide or zinc oxide are also possible, where the particle size of these pigments are greater than or equal to 200 nm, for example Hombitan® FG or Hombitan® FF-Pharma.

It may furthermore be preferred for the compositions to comprise inorganic UV filters which have been aftertreated by conventional methods, as described, for example, in Cosmetics & Toiletries, February 1990, Vol. 105, pp. 53-64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa,
aftertreated micronised titanium dioxides with aluminium oxide and silicon dioxide aftertreatment, such as, for example, the product “Microtitanium Dioxide MT 100 SA from Tayca; or the product “Tioveil Fin” from Uniqema,
aftertreated micronised titanium dioxides with aluminium oxide and/or aluminium stearate/laurate aftertreatment, such as, for example, Microtitanium Dioxide MT 100 T from Tayca, Eusolex T-2000 from Merck,
aftertreated micronised titanium dioxides with iron oxide and/or iron stearate aftertreatment, such as, for example, the product “Microtitanium Dioxide MT 100 F” from Tayca,
aftertreated micronised titanium dioxides with silicon dioxide, aluminium oxide and silicone aftertreatment, such as, for example, the product “Microtitanium Dioxide MT 100 SAS”, from Tayca,
aftertreated micronised titanium dioxides with sodium hexametaphosphates, such as, for example, the product “Microtitanium Dioxide MT 150 W” from Tayca.

The treated micronised titanium dioxides employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3″ from Cardre,
polydimethylhydrogenosiloxanes, such as, for example, the product Microtitanium Dioxide USP Grade Hydrophobic” from Color Techniques.

The combination with the following products may furthermore also be advantageous:
untreated zinc oxides, such as, for example, the product Z-Cote from BASF (Sunsmart), Nanox from Elementis
aftertreated zinc oxides, such as, for example, the following products:
   “Zinc Oxide CS-5” from Toshibi (ZnO aftertreated with polymethylhydrogenosiloxanes)
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilesquioxane);
   untreated cerium oxide micropigment, for example with the name “Colloidal Cerium Oxide” from Rhone Poulenc
   untreated and/or aftertreated iron oxides with the name Nanogar from Arnaud.

For example, it is also possible to employ mixtures of various metal oxides, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide. zinc oxide mixtures, such as, for example, the product UV-Titan M261 from Sachtleben, in combination with the UV protection agent according to the invention.

These inorganic UV filters are generally incorporated into the compositions in an amount of 0.1 percent by weight to 25 percent by weight, preferably 2% by weight-10% by weight.

By combination of one or more of the said compounds having a UV filter action, the protective action against harmful effects of the UV radiation can be optimised.

The said UV filters can also be employed in encapsulated form. It is advantageous here for the capsules to be so small that they cannot be observed with the naked eye. In order to achieve the above-mentioned effects, it is furthermore necessary for the capsules to be sufficiently stable and the encapsulated active compound (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsule walls may also consist of PMMA. Capsules particularly preferably to be employed have walls which can be obtained by a sol-gel process, as described in the applications WO 00/09652, WO 00/72806 and WO 00/71084. Preference is in turn given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules are preferably present in compositions to be employed in accordance with the invention in amounts which ensure that the encapsulated UV filters are present in the composition in the above-indicated percent by weight ratios.

Preferred compositions may also comprise at least one further cosmetic active compound, for example selected from antioxidants, anti-ageing active compounds, anti-cellulite active compounds, self-tanning substances, skin-lightening active compounds, antimicrobial active compounds or vitamins.

The protective action of compositions against oxidative stress or against the effect of free radicals can be improved if the compositions comprise one or more antioxidants, the person skilled in the art being presented with absolutely no difficulties in selecting antioxidants which act suitably quickly or with a time delay.

There are many proven substances known from the specialist literature which can be used as antioxidants, for example amino acids (for example glycine, histidine, tyrosine, tryptophan) and derivatives thereof, imidazoles and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example contents of pmol to μmol/kg), and also (metal) chelating agents, (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the formulae A or B

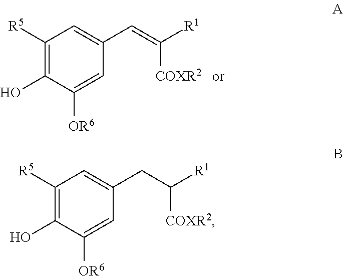

in which
R¹ can be selected from the group —C(O)CH₃, —CO₂R³, —C(O)NH₂ and —C(O)N(R⁴)₂,
X denotes O or NH,′
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes H or linear or branched alkyl having 1 to 8 C atoms or linear or branched alkoxy having 1 to 8 C atoms and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP). Furthermore, the combination with bisisopropyl 2-(4-hydroxy-3-methoxybenzylidene)malonate or bisisopropyl 2-(4-hydroxy-3-methoxybenzyl)malonate is preferred. An analogous situation applies to corresponding bisethyl esters.

The terms R¹ to R⁶ and X only apply here to the radicals of the formulae A and B and do not apply to the radicals of the formula I.

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid, natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such compositions with the compounds according to the invention in percent by weight ratios in the range from 1000:1 to 1:1000, preferably in percent by weight ratios of 100:1 to 1:100.

Of the phenols which can be used in accordance with the invention, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101, are concerned with effects of the substitution pattern of mono- and dihydroxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152.). K. Lemanska, H. Szymusiak, B. Tyrakowska, R. Zielinski, A. E. M. F. Soffers and I. M. C. M. Rietjens, Free Radical Biology Medicine 2001, 31(7), 869, have investigated the pH dependence of the antioxidant action of hydroxyflavones. Quercetin exhibits the highest activity amongst the structures investigated over the entire pH range.

Suitable anti-ageing active compounds, in particular for skin-care compositions, are preferably so-called compatible solutes. These are substances which are involved in the osmosis regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline and taurine.

Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidine-carboxylic acids (such as ectoin and hydroxyectoin), proline, betaine, glutamine, cyclic diphosphoglycerate, N.-acetylornithine, trimethylamine N-oxide di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidine-carboxylic acid) and derivatives thereof.

Furthermore, the compositions according to the invention may comprise at least one self-tanning agent as further ingredient.

Advantageous self-tanning agents which can be employed are, inter alia: 1,3-dihydroxyacetone, glycerolaldehyde, hydroxymethylglyoxal, γ-dialdehyde, erythrulose, 6-aldo-D-fructose, ninhydrin, 5-hydroxy-1,4-naphthoquinone (juglone) or 2-hydroxy-1,4-naphthoquinone (lawsone). Very particular preference is given to 1,3-dihydroxyacetone, erythrulose or a combination thereof.

The compositions may also comprise one or more further skin-lightening active compounds. Skin-lightening active compounds can in principle be all active compounds known to the person skilled in the art. Examples of compounds having skin-lightening activity are hydroquinone, kojic acid, arbutin, aloesin, niacinamide, emblica, liquorice exract, vitamin C, magnesium ascorbyl phosphate or rucinol.

The compositions to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the compositions in ranges from 0.01 to 5.0% by weight, based on the total weight. Nutrition-physiological applications are oriented towards the respective recommended vitamin requirement.

The retinoids described are at the same time also effective anti-cellulite active compounds. A likewise known anti-cellulite active compound is caffeine.

The said constituents of the composition can be incorporated in the usual manner, with the aid of techniques which are well known to the person skilled in the art.

Suitable compositions are those for external application, for example can be sprayed onto the skin as cream or milk (O/W, W/O, O/W/O, W/O/W), as lotion or emulsion, in the form of oily-alcoholic, oily-aqueous or aqueous-alcoholic gels or solutions. They can be in the form of solid sticks, as plasters or formulated as an aerosol. Administration forms such as capsules, dragees, powders, tablet solutions or solutions are suitable for internal use.

Examples which may be mentioned of application forms of the compositions to be employed are: solutions, suspensions, emulsions, PIT emulsions, pastes, ointments, gels, creams, lotions, powders, soaps, surfactant-containing cleansing preparations, oils, aerosols and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexanecarbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred composition forms also include, in particular, emulsions.

Emulsions are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, iso-propyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the compositions to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other fatty substances, as well as water and an emulsifier, as usually used for a formulation of this type.

In a preferred embodiment, the compositions to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The cosmetic and dermatological compositions may exist in various forms. Thus, they may be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A-43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alchols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth 1-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The composition may comprise cosmetic adjuvants which are usually used in this type of composition, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty substance, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, use is generally made of the customary propellants, preferably alkanes.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference. The percent by weight ratios of the individual ingredients in the compositions of the examples expressly belong to the disclosure content of the description and can therefore be utilised as features.

Further important features and advantages of the invention arise from the sub-claims, from the drawing, from the associated description of the figures with reference to the drawing and from the examples.

It goes without saying that the features mentioned above and still to be explained below can be used not only in the respective combination indicated, but also in other combinations or in isolation without leaving the framework of the present invention.

Preferred embodiments of the invention are depicted in the drawing and described in the examples and are explained in greater detail in the following description without restricting the scope of the present invention.

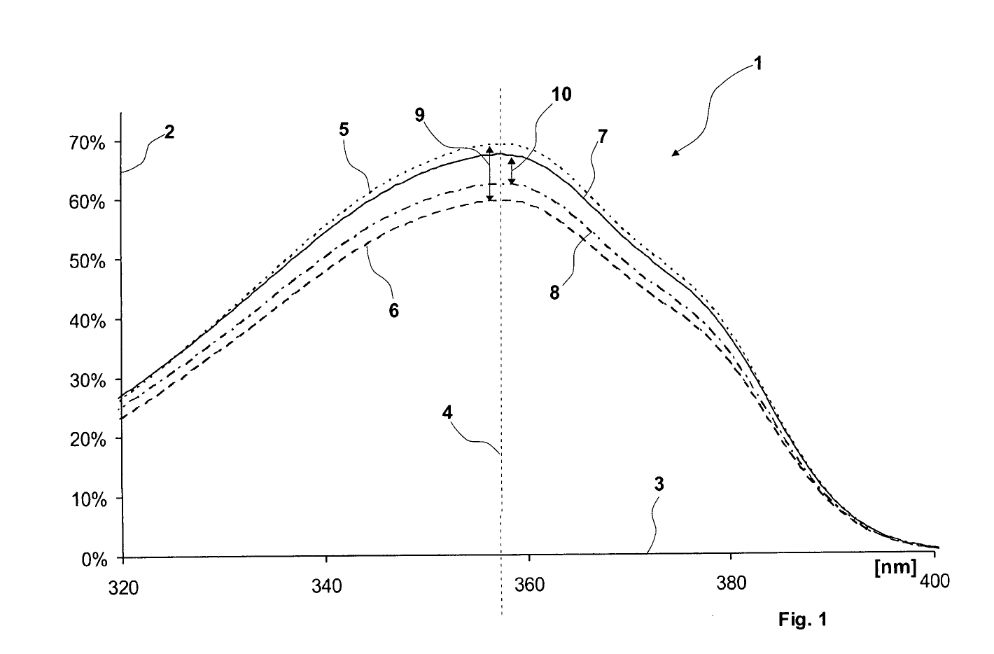

FIG. 1 illustrates the effect of the addition of 4-methoxyacetophenone as compound of the formula I to a solution of avobenzone as compound to be stabilised in dimethylisosorbide, commercially available under the name Arlasolv® DMI from Uniqema.

FIG. 1 depicts an absorption/wavelength diagram 1 with a plurality of absorption curves of a 2% by weight solution of avobenzone in Arlasolv® DMI. The absorption in % is plotted on the Y axis 2 in the absorption/wave diagram 1, while the wavelength in nm is plotted on the X axis 3. The absorption maximum of avobenzone at 357 nm is indicated in the form of a straight line 4 parallel to the Y axis 2 in the absorption/wavelength diagram 1. This straight line 4 can be used to make a prediction of the amount of avobenzone present, since, at a wavelength of 357 nm, the radiation of this wavelength in the solutions used is absorbed exclusively by avobenzone. 4-Methoxyacetophenone exhibits no absorption capacity in a wavelength region around 357 nm. In order to be able to make a prediction of how much avobenzone can be protected against photochemical decomposition by means of addition of 4-methoxyacetophenone on irradiation with sun-like light, an absorption curve 5 of an unirradiated two % by weight solution of avobenzone in Arlasolv DMI, an absorption curve 6 of an irradiated two % by weight solution of avobenzone in Arlasolv DMI, an absorption curve 7 of an unirradiated, in each case two % by weight solution of avobenzone and 4-methoxyacetophenone and an absorption curve 8 of an irradiated, in each case two % by weight solution of avobenzone and 4-methoxyacetophenone in Arlasolv DMI are shown in the absorption/wavelength diagram 1. The respective solutions of avobenzone or avobenzone and 4-methoxyacetophenone were in each case irradiated in the solvent Arlasolv DMI with a radiation dose of 325 kJ/m², which corresponds to an irradiation of 6.5 MED (minimal erythemal dose). A radiation having a spectral composition analogous to sunlight, in particular generated by a solar simulator, is preferably used.

As revealed by the absorption/wavelength diagram 1, absorption curves 5 and 7 of the unirradiated solutions, in particular at the absorption maximum of avobenzone of 357 nm or in the region of the straight line 4, in each case have higher absorption than absorption curves 6 and 8 of the irradiated solutions. Furthermore, it is evident through comparison of absorption curves 5 and 7 of the unirradiated solutions that admixing 4-methoxyacetophenone slightly reduces the absorption capacity of the solution.

If a pure solution of avobenzone is irradiated, depicted in absorption curves 5 and 6, it is evident that the absorption capacity of a pure avobenzone solution of this type is reduced by an absorption delta 9 of 9% after irradiation with a radiation dose described above. This absorption delta 9 at the absorption maximum of avobenzone at 357 nm is determined, for example, by means of the straight line 4. It can be concluded from this that irradiation of this pure avobenzone solution with radiation dose outlined above removes a significant part of the avobenzone from the solution through photochemical decomposition, since the absorption in the region of the straight line 4 is essentially caused by avobenzone.

If absorption curves 7 and 8 of the avobenzone/4-methoxyacetophenone solution before and after irradiation with the radiation dose described above are compared, it is evident that only an absorption delta 10 of 4% becomes established after irradiation. It can be concluded from this that admixing 4-methoxyacetophenone with a pure avobenzone solution enables the photochemical decomposition of avobenzone on irradiation with sunlight or sun-like light to be reduced at least partly, but still significantly compared with a pure avobenzone solution.
Ex. 1 shows the photochemical decomposition of dibenzoylmethanes, in particular avobenzone, by means of a decomposition scheme,
Ex. 2 shows S₁ and T₁ energy levels of a plurality of selected compounds of the formula I,
Ex. 3 shows S₁ and T₁ energy levels of a plurality of selected compounds to be stabilised,
Ex. 4 shows a plurality of illustrative cosmetic compositions comprising at least one compound of the formula I and a compound to be stabilised.

### EXAMPLE 1

### Photochemical Decomposition of Dibenzoylmethanes, in Particular Avobenzone

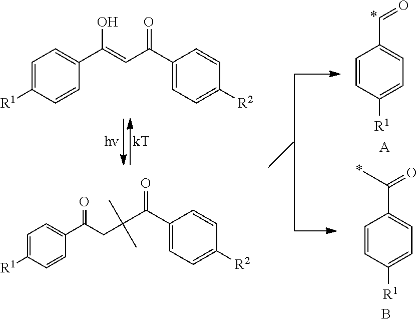

R¹ and R² here denote R¹¹ or OR¹¹, as described in detail above or described as preferred. In the case of avobenzone, R¹=methoxy and R²=tert-butyl.

Dibenzoylmethane derivatives, such as, for example, avobenzone, as example of a compound to be stabilised are usually in their enol form under normal conditions. These dibenzoylmethane derivatives can be converted into the diketo form, as one appearance form, by excitation by means of radiation. The diketo form can be converted back into the enol form by thermal relaxation. However, if the diketo form absorbs further radiation energy, it can react in accordance with a Norish type I reaction to give a benzoyl free radical (A) and a phenacyl free radical (B), where these free radicals can react further by recombination or other reactions. It can be assumed that the diketo form of the dibenzoylmethane derivatives is raised to its first excited triplet state T₁ by absorption of further radiation energy. This excited first triplet state of the diketo form tends towards Norish type I cleavage. In the case of avobenzone, the energy level of the first excited triplet state T₁ of the diketo form can be determined as 3.10 eV. The energy level of the first excited triplet state T₁ of 4-methoxacetophenone can be calculated as 3.15 eV. The two energy levels of the respective first excited triplet state T₁ are thus so closely adjacent to one another (1.6% difference) that the compound avobenzone to be stabilised can in its diketo form carry out a triplet-triplet energy exchange with the first compound 4-methoxyacetophenone, so that the compound avobenzone to be stabilised can be photostabilised in its diketo appearance form, which tends towards photochemical decomposition, by the compound 4-methoxyacetophenone. Thus, as shown in this example, the compound of the formula I, 4-methoxyacetophenone, is used as photostabiliser specifically for the appearance form, the diketo form, of the compound avobenzone to be stabilised whose first excited triplet state T₁ tends towards photoinstability.

### EXAMPLE 2

### S₁ and T₁ Energy Levels of a Plurality of Selected First Compounds

| First compound | S1 [eV] | T1 [eV] |
|---|---|---|
| 4-Methhoxyacetophenone | 3.71 | 3.15 |
| 4-Methoxybenzaldehyde | 3.69 | 3.05 |
| Benzaldehyde | 3.59 | 3.01 |
| 4-tert-Butylbenzaldehyde | 3.62 | 3.04 |
| Acetophenone | 3.61 | 3.05 |
| 4-tert-Butylacetophenone | 3.64 | 3.07 |
| 1-Methoxy-4-(1-methoxyvinyl)benzene | 4.60 | 3.10 |
| [1-(4-Methoxyphenyl)vinyloxy]trimethylsilane | 4.57 | 2.98 |
| 3-Acetyl-4-aminobenzonitrile | 3.38 | 2.92 |
| 4-Formyl-2-methylbenzonitrile | 3.73 | 2.82 |
| 1-(3,5-Di-tert-Butyl-4-hydroxyphenyl)ethanone | 3.73 | 3.02 |
| 1-(2,4-Dimethylphenyl)ethanone | 3.67 | 3.08 |
| 2-Acetyl-4-aminobenzonitrile | 3.69 | 2.95 |

### EXAMPLE 3

### S₁ and T₁ Energy Levels of a Plurality of Selected Second Compounds

| Second compound | S1 [eV] | T1 [eV] |
|---|---|---|
| Avobenzone | 3.37 | 3.10 |
| Uvinul T 150 | 3.93 | 3.16 |
| Tinosorb S | 3.47 | 2.96 |
| Uvasorb HEB | 3.92 | 3.16 |

### EXAMPLE 4

### Cosmetic Compositions

The following tables show by way of example recipes for cosmetic composition which comprise at least one compound of the formula I and at least one compound to be stabilised. Corresponding compositions can be prepared in the same way with all compound pairs according to the invention, comprising at least one compound of the formula I and at least one compound to be stabilised. Compositions comprising at least one such compound of the formula I and at least one such compound to be stabilised may in addition comprise further cosmetic ingredients, which are described in the specification WO 2009/098139, in any desired combination with one another.

### COMPOSITION EXAMPLES

Composition examples shown below may furthermore comprise the following stabilisers (each 0.1-10% in individual component or in mixtures): Benzotriazolyl Dodecyl p-Cresol (Tinoguard TL), Butyloctyl salicylate, Diethylhexyl 2,6-Naphthalate, Diethylhexyl Syringylidene malonate, Polyester-8 (Polycrylene), bis-Ethylhexyl Hydroxydimethoxy Benzylmalonate.

### EXAMPLE 1

### W/O Emulsion

| | 54-07- | 54-07- | 54-07- | 54-07- | 54-07- |
|---|---|---|---|---|---|
| | 5-A | 5-B | 5-C | 5-D | 5-E |
| Cetyl PEG/PPG-10/1 | 3.00 | 3.00 | 3.00 | 3.00 | 3.00 |
| dimethicone | | | | | |
| (Abil EM 90) | | | | | |
| Polyglyceryl-4 | 1.50 | 1.50 | 1.50 | 1.50 | 1.50 |
| isostearate | | | | | |
| (Isolan GI 34) | | | | | |
| Butylphthalimide | 5.00 | 5.00 | 5.00 | 5.00 | 5.00 |
| isopropylphthal- | | | | | |
| imide | | | | | |
| (Pelemol ® BIP) | | | | | |
| Dimethyl isosorbide | 5.00 | 5.00 | 5.00 | 5.00 | 5.00 |
| (Arlasolve DMI) | | | | | |
| 4-Methoxy- | 1.00 | | | 1.00 | 2.00 |
| acetophenone | | | | | |
| Uvinul ® A Plus | | 0.84 | 0.84 | 1.00 | |
| Uvasorb Heb | 2.0 | 2.0 | 1.5 | 4.0 | 5.0 |
| Tinosorb S | 2.0 | 2.0 | 3.0 | 1.5 | |
| Ascorbic acid | | | 0.37 | 1.00 | 3.00 |
| Mineral Oil | 8.00 | 8.00 | 8.00 | 8.00 | 8.00 |
| Ethylhexyl stearate | 5.00 | 5.00 | 5.00 | 5.00 | 5.00 |
| (Tegosoft ® OS) | | | | | |
| Cyclomethicone (and) | 5.00 | 5.00 | 5.00 | 5.00 | 5.00 |
| Aluminium/Magnesium | | | | | |
| Hydroxide Stearate | | | | | |
| (Gilugel SIL 5) | | | | | |
| Preservative | 1.00 | 1.00 | 1.00 | 1.00 | 1.00 |
| Water | To 100 | To 100 | To 100 | To 100 | To 100 |
| NaCl | 0.50 | 0.50 | 0.50 | 0.50 | 0.50 |
| EDTA | 0.10 | 0.10 | 0.10 | 0.10 | 0.10 |
| Citric acid q.S. | | | | | |

Preparation: Pelemol BIP, Arlasolv DMI and emulsifiers are initially introduced. 3-(4-tert-Butylphenyl)-3-hydroxy-1-(4-methoxyphenyl)propan-1-one, 3-(4-methoxy)-3-hydroxy-1-(4-tert-butylphenyl)propan-1-one, Uvinul® A Plus and the trazines are dissolved therein. The remaining constituents of the oil phase are added and mixed homogeneously. The water phase, adjusted to pH=4-5, is emulsified in with stirring. The mixture is subsequently homogenised. The emulsions can be prepared under gentle conditions at room temperature.

### EXAMPLE II

### Water-Resistant Sunscreen Spray

| A | | | |
|---|---|---|---|
| 4-Methoxyacetophenone | 1.00 | 1.00 | 2.00 |
| Diethylhexyl | | 0.50 | |
| Syringylidenemalonate, | | | |
| Caprylic/Capric Triglyceride | | | |
| (Oxynex ® ST Liquid) | | | |
| RonaCare ® AP | | 2.00 | |
| Tinsorb S | 1.5 | 1.0 | 1.5 |
| Uvasorb Heb | 1.5 | 3.0 | 5.0 |
| Uvinul T150 | 1.5 | 0.5 | |
| Ascorbyl Palmitate | | | 1.00 |
| Cyprylic/capric Triglyceride | 7.00 | 7.00 | 7.00 |
| (Miglyol 812 N) | | | |
| Butylphthalimide | 10.00 | 10.00 | 10.00 |
| isopropylphthalimide | | | |
| (Pelemol ® BIP) | | | |
| C12-15 alkyl benzoate | 10.00 | 10.00 | 10.00 |
| (Tegosoft ® TN) | | | |
| Phenethyl benzoate | 5.00 | 5.00 | 5.00 |
| (X-Tend 226) | | | |
| RonaCare ® tocopherol acetate | 1.00 | 1.00 | 1.00 |
| B | | | |
| Cyclopentasiloxane | 43.80 | 41.30 | 41.80 |
| (Dow Corning 245) | | | |
| Phenyltrimethicone | 2.00 | 2.00 | 2.00 |
| (Dow Corning 556) | | | |
| Cyclopentasiloxane, | 20.00 | 20.00 | 20.00 |
| dimethiconol | | | |
| Dow Corning 1501 Fluid | | | |
| Perfume oil (q.s.) | 0.20 | 0.20 | 0.20 |

Preparation: the components of phase A are combined at room temperature and stirred. Phase B is subsequently mixed and added to phase B with stirring, and the mixture is stirred.

### EXAMPLE III

### Pump Hairspray

| | | | |
|---|---|---|---|
| A | | | |
| 4-Methoxyacetophenone | 1.00 | 2.00 | 4.00 |
| Butylmethoxydibenzoylmethane | 1.00 | 1.00 | 1.00 |
| Uvasorb Heb | | 1.00 | |
| Ethanol 96% extra pure | To 100 | To 100 | To 100 |
| PVP/VA copolymer | 6.00 | 6.00 | 6.00 |
| PVP/VA W 735 | | | |
| B | | | |
| Diethylhexyl Syringylidenemalonate, | 0.06 | 0.25 | 0.50 |
| Caprylic/Capric Triglyceride | | | |
| (Oxynex ® ST Liquid) | | | |
| PEG-75 Lanolin | 0.20 | 0.20 | 0.20 |
| BHT | | | |
| (Solan E - Low Dioxane) | | | |
| Perfume | 0.10 | 0.10 | 0.10 |
| (Frag 280853 Green Activating) | | | |
| C | | | |
| Water, demineralised | 13.00 | 13.00 | 13.00 |
| Titriplex III | 0.10 | 0.10 | 0.10 |
| PEG-12 dimethicone | 0.50 | 0.50 | 0.50 |
| Dow Corning 193 Fluid | | | |
| 0.1% D&C Red No 33 (CI 17200) | 0.20 | 0.20 | 0.20 |
| in water | | | |
| PEG-40 Hydrogenated Castor Oil | 1.00 | 1.00 | 1.00 |
| (Cremophor RH410) | | | |

Preparation: pre-dissolve phase A. Add phase B to phase A with stirring.

Pre-mix phase C and add to the remainder, stir until a homogeneous

### EXAMPLE IV

### W/O Emulsions

| Emulsion | A | B | C | D | E | F |
|---|---|---|---|---|---|---|
| Polyglyceryl 2-dipoly- | 3 | 5 | 3 | | | |
| hydroxystearate | | | | | | |
| PEG-30 Dipolyhydroxy- | | | 2 | 3 | 4 | 5 |
| stearate | | | | | | |
| Sodium starch | 0.5 | 0.4 | | 0.3 | | 1 |
| octenylsuccinate | | | | | | |
| Glycine | 0.3 | 0.3 | 0.5 | 0.4 | | |
| Alcohol | | 5 | 2 | 5 | 4 | |
| Magnesium sulfate | 0.2 | 0.3 | 0.3 | 0.4 | 0.5 | 0.2 |
| C₁₂₋₁₅ alkyl benzoate | 5 | 3 | | | 5 | |
| C₁₂₋₁₃ alkyl tartrate | | 2 | | | | |
| Butylene glycol | 5 | | | | 3 | 3 |
| dicaprylate/dicaprate | | | | | | |
| Dicaprylyl ether | | | | | 2 | |
| Mineral oil | | 4 | | 6 | | 8 |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene glycol | | | 1 | | 5 | 3 |
| Glycerine | 3 | 5 | 7 | 10 | 3 | 3 |
| C18-38 acid triglycerides | 0.5 | | 1 | | 1 | |
| Titanium dioxide | 5 | 6 | 4 | | | 4 |
| Zinc oxide | 5 | | | | | |
| Tinsosorb S | | 3 | 3 | 2 | | |
| Uvinul T150 | | 4.5 | 3 | | 3 | |
| 4-Methoxyacetophenone | 2.0 | 0.5 | 1.0 | 1.0 | 3.0 | 1.0 |
| Uvasorb Heb | | | 1.5 | 4 | | |
| Butylmethoxydibenzoyl- | 2 | 3 | 4 | | 1 | 3 |
| methane | | | | | | |
| Uvinul ® A Plus | | | | 4 | 2 | |
| Ethylhexyl methoxycinnamate | | | | | 7 | 5 |
| Benzotriazole coupled to | 4 | | 6 | | | |
| gelatine | | | | | | |
| Taurine | 0.1 | | | 0.5 | 0.2 | |
| Vitamin E acetate | 0.2 | 02 | | 0.3 | 0.1 | 0.5 |
| Na₂H₂EDTA | 0.1 | 0.1 | 0.2 | 0.2 | 0.2 | 0.5 |
| C8-C16 alkylpolyglycoside | 1 | | | | | |
| Perfume, preservatives | q.s. | q.s | q.s | q.s | qs. | qs. |
| Dyes, etc. | q.s. | q.s. | q.s. | q.s | q.s. | q.s. |
| Sodium hydroxide | q.s. | q.s. | q.s. | q.s | q.s. | q.s. |
| Water | to | to | to | to | to | to |
| | 100.0 | 100.0 | 100.0 | 100.0 | 100.0 | 100.0 |

### EXAMPLE V

### Hair-Care Formulation

Content in g of component per 100 g of formulation

| Component | A | B | C | D | E | F |
|---|---|---|---|---|---|---|
| Disodium EDTA | 0.100 | 0.100 | 0.100 | 0.100 | 0.100 | 0.100 |
| Oxynex ® ST | 2.000 | 2.000 | 2.000 | 2.000 | 2.000 | 2.000 |
| 4-Methoxyacetophenone | 1.5 | 0.25 | 0.50 | 1.50 | 2.00 | 4.00 |
| Uvinul T150 | 0.50 | 0.50 | 0.50 | 0.50 | 0.50 | 0.50 |
| TinosorbA2B | 0.50 | 0.50 | 0.50 | 0.50 | 0.50 | 0.50 |
| Hexamidine diisothionate | 0.100 | 0 | 0 | 0 | 0 | 0 |
| Tetrahydrocurcumine | 0 | 0.500 | 0 | 0 | 0 | 0 |
| Glycyrrhetinic acid | 0 | 0 | 0.300 | 0 | 0 | 0 |
| Thiotaine ®¹ | 0 | 0 | 0 | 5.000 | 0 | 0 |
| N-undecylenoyl-L-phenylalanine | 0 | 0 | 0 | 0 | 1.000 | 0 |
| N-acetyl glucosamine | 0 | 0 | 0 | 0 | 0 | 2.000 |
| Niacinamide | 5.000 | 5.000 | 5.000 | 5.000 | 5.000 | 5.000 |
| Citric acid | 0.015 | 0 | 0 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 5.000 | 0 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl | 0.200 | 0.200 | 0.200 | 0.200 | 0.200 | 0.200 |
| alcohol | | | | | | |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerine | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 | 3.000 | 2.000 | 2.000 | 2.000 | 2.000 | 2.000 |
| isoparaffin + laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

### EXAMPLE VI

### Hair-Care Formulation

Content in g of component per 100 g of formulation

| Component | G | H | I |
|---|---|---|---|
| Disodium EDTA | 0.100 | 0.100 | 0.100 |
| Oxynex ® ST | 2.000 | 2.000 | 2.000 |
| Tinsorb S | 1.00 | 1.00 | 1.00 |
| Uvinul T150 | 1.00 | 1.00 | 1.00 |
| 4-Methoxyacetophenone | 2.00 | 3.50 | 1.50 |
| Cetyl pyridinium chloride | 0.200 | 0 | 0 |
| Pitera ® | 0 | 10 | 0 |
| Ascorbyl glycoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl isostearate | 1.330 | 1.330 | 1.330 |
| Sucrose polycottonseedate | 0.670 | 0.670 | 0.670 |
| Polymethylsilsesquioxane | 0.250 | 0.250 | 0.250 |
| Cetearyl glucoside + cetearyl alcohol | 0.200 | 0.200 | 0.200 |
| Behenyl alcohol | 0.400 | 0.400 | 0.400 |
| Ethylparaben | 0.200 | 0.200 | 0.200 |
| Propylparaben | 0.100 | 0.100 | 0.100 |
| Cetyl alcohol | 0.320 | 0.320 | 0.320 |
| Stearyl alcohol | 0.480 | 0.480 | 0.480 |
| Tocopheryl acetate | 0.500 | 0.500 | 0.500 |
| PEG-100 stearate | 0.100 | 0.100 | 0.100 |
| Glycerine | 7.000 | 7.000 | 7.000 |
| Titanium dioxide | 0.604 | 0.604 | 0.604 |
| Polyacrylamide + C13-14 isoparaffin + | 2.000 | 2.000 | 2.000 |
| Iaureth-7 | | | |
| Panthenol | 1.000 | 1.000 | 1.000 |
| Benzyl alcohol | 0.400 | 0.400 | 0.400 |
| Dimethicone + dimethiconol | 2.000 | 2.000 | 2.000 |
| Water (to 100 g) | to 100 | to 100 | to 100 |
| TOTAL | 100 | 100 | 100 |

### EXAMPLE VII

### O/W Emulsions

| Emulsion | A | B | C | D | E | F |
|---|---|---|---|---|---|---|
| Glyceryl stearate citrate | 2.5 | 2 | 3 | | | |
| Sorbitan stearate | 0.5 | | | 2 | 1.5 | 2 |
| Polyglyceryl-3 methylglycose | | | | 2.5 | 3 | 3 |
| distearate | | | | | | |
| Polyglyceryl-2 | | 0.8 | | | | 0.5 |
| dipolyhydroxystearate | | | | | | |
| Cetearyl alcohol | | | | 1 | | |
| Stearyl alcohol | 2 | | | | | 2 |
| Cetyl alcohol | | 1 | | | 3 | |
| Acrylates/C₁₀₋₃₀ alkyl acrylate | | 0.2 | | | 0.1 | |
| crosspolymer | | | | | | |
| Carbomer | | 0.2 | 0.3 | 0.2 | | |
| Xanthan Gum | 0.4 | | 0.2 | 0.2 | 0.3 | 0.4 |
| C₁₂₋₁₅ alkyl benzoate | 5 | 3 | | | 5 | |
| C₁₂₋₁₃ alkyl tartrate | | 2 | | | | |
| Butylene glycol | 5 | | | | 3 | 3 |
| dicaprylate/dicaprate | | | | | | |
| Dicaprylyl Ether | | | | | 2 | |
| Octyldodecanol | 2 | | | | | |
| Dicapryl caprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene gykol | | | 1 | | 5 | 3 |
| Glycerine | 3 | 5 | 7 | 10 | 3 | 3 |
| C18-C38 acid triglycerides | 0.5 | | 1 | | 1 | |
| Titanium dioxide | 5 | | | 2 | | |
| 2,2′-Methylenebis(6-(2H- | 2.5 | | | | | |
| benzotriazol-2-yl)-(1,1,3,3- | | | | | | |
| tetramethylbutyl)phenol) | | | | | | |
| Tinosorb A2B | | 2 | | | | |
| Uvasorb Heb | 4 | | 3 | | 3 | 2 |
| Benzotriazole coupled to | | 5 | | 10 | | 3 |
| gelatine | | | | | | |
| C8-C16 alkylpolyglycoside | 1 | 0.6 | | | | |
| Uvasorb K2A | | | 2 | | | |
| Uvinul ® A Plus | 2 | | | | | 1 |
| Homosalate | | 5 | | 1 | | |
| Phenylbenzimidazolesulfonic | | | 2 | | | 1 |
| acid | | | | | | |
| Benzophenone-3 | 2 | | | | 2 | |
| Octyl salicylate | 5 | 5 | | 2 | | |
| Octocrylene | 2 | | | | 3 | 1 |
| 4-Methoxyacetophenone | 1.0 | 2.0 | 3.0 | 1.0 | 2.0 | 3.0 |
| Tinsosorb S | 1.2 | 3 | 2 | 1 | | |
| Parsol ® SLX | | | 3 | | | |
| Dihydroxyacetate | | | | | 4 | |
| Taurine | 0.1 | | | 0.5 | 0.2 | |
| 8-Hexadecene-1,16- | | 0.2 | | | | |
| dicarboxylic acid | | | | | | |
| Vitamin E acetate | 0.2 | 0.2 | | 0.3 | 0.1 | 0.5 |
| Na₂H₂EDTA | 0.1 | 0.1 | 0.2 | 0.2 | 0.2 | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to | to | to | to | to | to |
| | 100.0 | 100.0 | 100.0 | 100.0 | 100.0 | 100.0 |

### EXAMPLE VIII

### O/W Emulsions

| Emulsion | G | H | I | K | L | M |
|---|---|---|---|---|---|---|
| Ceteareth-20 | 1 | 1.5 | 1 | | | |
| Sorbitan stearate | | | 0.5 | | 0.5 | |
| Glyceryl stearate SE | | | | 1 | 1 | 1.5 |
| Emulgade F ® | | | | 2.5 | 2.5 | 3 |
| Cetearyl alcohol | | | | 1 | | |
| Stearyl alcohol | | | | | 1.5 | |
| Cetyl alcohol | | | 0.5 | | | 2 |
| Acrylates/C₁₀₋₃₀ alkyl acrylate | 0.2 | 0.4 | 0.3 | 0.1 | | |
| crosspolymer | | | | | | |
| Carbomer | | | | | 0.3 | |
| Xanthan Gum | | | | 0.4 | | 0.4 |
| C₁₂₋₁₅ alkyl benzoate | 5 | 3 | | | 5 | |
| 2-Phenyl benzoate | | 2 | | | | |
| Butylene glycol | 5 | | | | 3 | 2 |
| dicaprylate/dicaprate | | | | | | |
| Dicaprylyl Ether | | | | | 2 | |
| Diethylhexyl naphthalate | 2 | | | | | |
| Dicapryl caprate | | 2 | | | 2 | 2 |
| Cyclomethicone | 5 | | 5 | 10 | | |
| Isohexadecane | | | | 5 | | |
| Mineral oil | | 1 | | | | |
| Propylene glycol | | | 4 | | | |
| Glycerine | 5 | 7 | 3 | 5 | 6 | 8 |
| C18-38 acid triglyceride | 0.5 | | 1 | | 1 | |
| Titanium dioxide | 5 | | 3 | 2 | | |
| NeoHeliopan ® AP | | 2 | | | 1 | 1 |
| Phenylbenzimidazolesulfonic | 1 | | | 1 | 2 | 1 |
| acid | | | | | | |
| Ethylhexyl methoxycinnamate | 5 | | 4 | 4 | | |
| Uvinul T150 | | 2 | | 1 | | |
| Uvasorb Heb | 1 | | | | 3 | |
| Butylmethoxydibenzoyl- | 2.5 | | 2 | 2 | | 1 |
| methane | | | | | | |
| Tinsosorb S | 2 | 0.5 | 1.5 | | | 2.0 |
| 4-Methylbenzylidenecamphor | 3 | | | | | |
| Parsol ® SLX | | | | | 2 | |
| 4-Methoxyacetophenone | 1.0 | 2.0 | 4.0 | 5.0 | 1.5 | 3.0 |
| Creatinine | 0.1 | 0.01 | 0.05 | | | |
| Creatine | 0.5 | 0.2 | 0.1 | | | |
| Liquorice extract/licochalcone | | | | 0.5 | | |
| Vitamin E acetate | 0.2 | | | 0.5 | 0.5 | 0.5 |
| Tapioca starch | | 3 | | | 2 | |
| Na₂H₂EDTA | 0.1 | | 0.2 | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to | to | to | to | to | to |
| | 100.0 | 100.0 | 100.0 | 100.0 | 100.0 | 100.0 |

### EXAMPLE IX

### O/W Emulsions

| Emulsion | N | O | P | Q | R | S |
|---|---|---|---|---|---|---|
| Glyceryl stearate SE | | 2 | | 2 | | |
| Glyceryl stearate | 2 | | 2 | | | |
| PEG-40 stearate | | | 2 | | 1 | |
| PEG-10 stearate | | | | 2.5 | 1 | |
| Ceteareth-20 | | | | | | 2.6 |
| Sodium cetyl phosphate | | | | | 2 | |
| Glyceryl stearate, | | | | | | 5.4 |
| ceteareth-12, ceteareth-20, | | | | | | |
| cetearyl alcohol, cetyl | | | | | | |
| palmitate | | | | | | |
| Stearic acid | 3 | 2 | | | 2 | |
| Stearyl alcohol | | 2 | 2 | | | |
| Stearyl alcohol | 0.5 | | 2 | | | |
| Cetyl alcohol | 3 | | | 2 | | |
| Acrylates/C₁₀₋₃₀ alkyl acrylate | | | 0.2 | | 0.4 | |
| crosspolymer | | | | | | |
| Carbomer | | 0.3 | | 0.3 | 0.3 | |
| Xanthan Gum | | 0.3 | 0.4 | | | |
| C₁₂₋₁₅ alkyl benzoate | 5 | | | | 5 | 3 |
| 2-Phenyl benzoate | 5 | | | | | |
| Butylene glycol | | 5 | | 4 | | 3 |
| dicaprylate/dicaprate | | | | | | |
| Dicaprylyl Ether | | 2 | | | 3 | |
| Diethylhexyl naphthalate | 3 | | | | | |
| Cyclomethicone | 2 | | 10 | 2 | | |
| Isohexadecane | | | | 2 | 3 | |
| Mineral oil | | | | | 3 | |
| Propanediol | | 3 | | 5 | | |
| Glycerine | 3 | 5 | 10 | 7 | 4 | 5 |
| Titanium dioxide | 2 | 4 | | | | |
| Zinc oxide | | | | | 2 | |
| Drometrizole Trisiloxane | | | | | 3 | |
| Ethylhexyl methoxycinnamate | | 6 | 5 | | | |
| Phenylbenzimidazolesulfonic | | 0.5 | 2 | | 1 | |
| acid | | | | | | |
| Homosalate | 5 | | | 7 | | |
| Butylmethoxydibenzoyl- | | 3 | | | | |
| methane | | | | | | |
| Tinsorb S | | 2 | 3 | | | |
| Uvasorb Heb | 1.5 | 1.5 | 1.5 | 1.5 | 1.5 | 1.5 |
| Octyl salicylate | | | | 5 | | |
| Octocrylene | 1.0 | | | | 3 | |
| 4-Methoxyacetophenone | 3.0 | 1.5 | 0.5 | 2.5 | 1.0 | 5.0 |
| Parsol ® SLX | 4 | | | | | 5 |
| PVP hexadecene copolymer | 0.5 | | 1 | | 0.8 | |
| Coenzyme Q 10 | 0.2 | 0.02 | | 0.3 | | |
| Vitamin E acetate | 0.2 | | 0.3 | | 0.8 | 0.5 |
| Na₂H₂EDTA | 0.1 | | | | | 0.5 |
| Perfume, preservatives | q.s. | q.s. | q.s. | q.s. | q.s. | q.s. |
| Dyes, etc. | q.s. | q.s. | q.s. | q.s. | q.s. | q.s. |
| Sodium hydroxide | q.s. | q.s. | q.s. | q.s. | q.s. | q.s. |
| Water | to | to | to | to | to | to |
| | 100.0 | 100.0 | 100.0 | 100.0 | 100.0 | 100.0 |

### EXAMPLE X

### Hydrodisperions (Lotions and Sprays)

| | A | B | C | D | E | F |
|---|---|---|---|---|---|---|
| Glyceryl stearate citrate | | 0.40 | | | | |
| Cetyl alcohol | | | | | 2.00 | |
| Sodium carbomer | | | | | 0.30 | |
| Acrylates/C₁₀₋₃₀ Alkyl | 0.30 | | 0.30 | 0.40 | 0.10 | 0.10 |
| Acrylate Crosspolymer | | | | | | |
| Cetsareth-20 | | | 1.00 | | | |
| Xanthan gum | | | | 0.15 | | 0.50 |
| Dimethicone/Vinyl | | | | 5.00 | | 3.00 |
| Dimethicone Crosspolymer | | | | | | |
| Uvasorb K2A | | | | | 3.50 | |
| Uvinul ® A Plus | 0.25 | | | 0.50 | 2.00 | 1.50 |
| Butylmethoxydibenzoyl- | 1.20 | | 3.50 | | | |
| methane | | | | | | |
| Tinsosorb S | 2.00 | 2.00 | | 0.25 | | |
| Terephthalidenedicamphor- | | | | | | 0.50 |
| sulfonic acid | | | | | | |
| Disodium phenyl- | | | | | | 1.00 |
| dibenzimidazoletetrasulfonate | | | | | | |
| Phenylbenzimidazolesulfonic | | | 2.00 | | | |
| acid | | | | | | |
| Ethylhexyl methoxycinnamate | 5.00 | | 7.00 | | 5.00 | 8.00 |
| Uvasorb Heb | | | 2.00 | 2.00 | | 2.00 |
| Uvinul T150 | 4.00 | 3.00 | 0.5 | | 4.00 | |
| Octocrylene | | | | 10.00 | | 2.50 |
| 4-Methoxyacetophenone | 0.25 | 1.5 | 2.0 | 2.5 | 1.0 | 5.0 |
| C₁₂₋₁₅ alkyl benzoate | 2.00 | | 2.50 | | | |
| Phenethyl benzoate | 4.00 | | | 7.50 | | 5.00 |
| C₁₈₋₃₆ triglyceride fatty acid | | | 1.00 | | | |
| Butylene glycol | | | | | 6.00 | |
| dicaprylate/dicaprate | | | | | | |
| Dicaprylyl carbonate | | 3.00 | | | | |
| Dicaprylyl ether | | 2.00 | | | | |
| Cyclomethicone | | | | 1.50 | | |
| Lanolin | | | | | 0.35 | |
| PVP hexadecene copolymer | 0.50 | | 0.50 | | 0.50 | 1.00 |
| Ethylhexyloxyglycerine | | 0.75 | | 1.00 | | 0.50 |
| Glycerine | 10.00 | 5.00 | 5.00 | | 5.00 | 15.00 |
| Butylene glycol | | 7.00 | | | | |
| Glycine soya | | | | 1.00 | | |
| Vitamin E acetate | 0.50 | 0.25 | 0 50 | 0.25 | 0.75 | 1.00 |
| α-Glycosylrutin | | | | | 0.25 | |
| Trisodium EDTA | | 1.00 | 1.00 | 0.10 | 0.20 | |
| Idopropynyl butylcarbamate | 0.20 | 0.10 | | | | 0.15 |
| Methylparaben | 0.50 | | 0.20 | | 0.15 | |
| Phenoxyethanol | 0.50 | 0.40 | 0.40 | | 1.00 | 0.60 |
| Ethanol | 3.00 | 10.00 | 4.00 | 3.50 | | 1.00 |
| Perfume, dyes | q.s. | q.s. | q.s. | qs. | q.s. | q.s. |
| Water | to | to | to | to | to | to |
| | 100 | 100 | 100 | 100 | 100 | 100 |
| Neutralisers (sodium | qs | qs | qs | qs | qs | qs |
| hydroxide, potassium | | | | | | |
| hydroxide) | | | | | | |

### EXAMPLE XI

### Aqueous and Aqueous/Alcoholic Formulations

| | A | E | C | D | E | F |
|---|---|---|---|---|---|---|
| Ethanol | 50 | 5 | 2 | 40 | 15 | |
| Hydroxyethylcellulose | 0.5 | | | | | |
| Acrylates/C10-30 Alkyl | | | | 0.3 | 0.6 | |
| Acrylate Crosspolymer | | | | | | |
| Cocoatnidopropylbetaine | | | 0.3 | | | |
| Formula XV (Uvasorb K2A) | | | | | 2 | |
| Uvinul ® APlus | 5 | | | | | |
| Butylmethoxydibenzoyl- | 0.5 | | | 3 | | |
| methane | | | | | | |
| Disodium phenyldibenzimida- | | 2 | 1 | | | |
| zoletetrasulfonate | | | | | | |
| Phenylbenzimidazolesulfonic | | 5 | 3 | | 2 | 4 |
| acid | | | | | | |
| Ethylhexyl methoxycinnamate | 10 | | | | 3 | |
| Uvasorb Heb | | 1 | | 3 | | |
| Uvinul T150 | | 1 | 1 | | 2 | 1 |
| Octocrylene | | | | 5 | | |
| 4-Methoxyacetophenone | 2.5 | 3.0 | 1.5 | 3.0 | 3.5 | 4.0 |
| C₁₂₋₁₅ alkyl benzoate | | | | 3 | | |
| C18-36 triglyceride fatty acid | | | | 1 | | |
| Butylene glycol | 2 | | | | | |
| dicaprylate/dicaprate | | | | | | |
| C12-13 alkyl tartrate | | | | | 5 | |
| Cyclomethicone | 4 | | | 2 | | |
| Insect Repellent ® 3535 | | | | 5 | | |
| Dimethicone | | | | | 3 | |
| PVP hexadecene copolymer | | 0.5 | | 1 | | 0.5 |
| Ethylhexyloxyglycerine | | 0.5 | | | | |
| Glycerine | 5 | 7 | 3 | 8 | | S |
| Butylene glycol | | | 5 | | 5 | |
| Metylpropanediol | | | | 4 | | |
| Vitamin E acetate | | 0.3 | 0.2 | 0.5 | | |
| Panthenol | 0.5 | | 0.2 | | | 0.3 |
| Creatinine | | | 0.01 | | 0.02 | |
| Creatine | | | 0.1 | | 0.2 | |
| PEG-40 hydrogenated castor | | 0.5 | 0.3 | | | 0.5 |
| oil | | | | | | |
| Trisodium EDTA | 0.3 | 0.2 | 0.2 | 0.2 | 0.2 | 0.5 |
| Preservatives | q.s. | q.s. | q.s. | q.s. | q.s. | q.s. |
| Sodium hydroxide | q.s. | q.s. | q.s. | q.s. | q.s. | q.s. |
| Perfume, dyes | q.s. | q.s. | q.s. | q.s. | q.s. | q.s. |
| Water | to | to | to | to | to | to |
| | 100 | 100 | 100 | 100 | 100 | 100 |

### EXAMPLE XII

### Cosmetic Foams

| Emulsion | A | B | C |
|---|---|---|---|
| Stearic acid | 2 | 2 | |
| Palmitic acid | | | 1.5 |
| Cetyl alcohol | 2.5 | 2 | |
| Stearyl alcohol | | | 3 |
| PEG-100 stearate | | | 3.5 |
| PEG-40 stearate | | 2 | |
| PEG-20 stearate | 3 | | |
| Sorbitan stearate | | 0.8 | |
| C₁₂₋₁₅ alkyl benzoate | 5 | | |
| C₁₂₋₁₃ alkyl tartrate | | | 7 |
| Butylene glycol dicaprylate/dicaprate | | 6 | |
| Dicaprylyl Ether | | | 2 |
| Cyclomethicone | | 2 | 3 |
| Butylene glycol | 1 | | |
| Isohexadecane | 2 | | |
| Methylpropanediol | | | |
| Propylene glycol | | | 5 |
| Glycerine | 5 | 7 | |
| Uvasorb K2A | 0.5 | 0.5 | 2 |
| Uvinul ® A Plus | 2 | 3 | |
| 4-Methoxyacetophenone | 0.5 | 1.0 | 1.5 |
| Parsol SLX ® | | 3 | |
| Homosalate | | 5 | |
| Phenylbenzimidazolesulfonic acid | | 2 | 2 |
| Benzophenone-3 | 2 | | |
| Octyl salicylate | | 5 | |
| Octocrylene | 2 | | |
| Tinsosorb S | 2 | 3 | 2 |
| 2,2′-Methylenebis(6-(2H-benzotriazol- | | | 8 |
| 2-yl)-4-(1,1,3,3-tetramethylbutyl)- | | | |
| phenol) | | | |
| Tinosorb A2B | 5 | | 4 |
| C8-C16 alkylpolyglycosides | 1 | | |
| Vitamin E acetate | 0.6 | 0.5 | 0.2 |
| Creatine/Creatinine | | | 0.5 |
| BHT | | | 0.1 |
| Na₂H₂EDTA | 0.50 | | |
| Perfume, preservatives | q.s. | q.s. | q.s. |
| Dyes, etc. | q.s. | q.s. | q.s. |
| Sodium hydroxide | q.s. | | q.s. |
| Potassium hydroxide | | q.s. | |
| Water | to 100.0 | to 100.0 | to 100.0 |

### EXAMPLE XIII

### Cosmetic Foams

| Emulsion | D | E | F | G |
|---|---|---|---|---|
| Stearic acid | 2 | | | |
| Palmitic acid | | | 3 | 3 |
| Cetyl alcohol | 2 | 2 | | |
| Cetylstearyl alcohol | | | 2 | 2 |
| Stearyl alcohol | | | | |
| PEG-100 stearate | | 4 | | |
| PEG-40 stearate | 2 | | | |
| PEG-20 stearate | | | 3 | 3 |
| Sorbitan stearate | 0.8 | | | |
| Tridecyl Trimellitate | | 5 | | |
| C₁₂₋₁₅ alkyl benzoate | | | 3 | 3 |
| Butylene glycol | 8 | | | |
| dicaprylate/dicaprate | | | | |
| Octyldodecanol | | 2 | | |
| Cocoglyceride | | | | 2 |
| Dicaprylyl Ether | | | 2 | 2 |
| Cyclomethicone | | | | |
| Dimethicone | 1 | | 2 | 2 |
| Isohexadecane | | 3 | | |
| Methylpropanediol | | 4 | | |
| Propylene glycol | | | | |
| Glycerine | 5 | | 6 | 6 |
| NeoHeliopan ® AP | | 2 | | |
| Phenylbenzimidazolesulfonic | 1 | | | 1 |
| acid | | | | |
| 4-Methoxyacetophenone | 2.0 | 1.5 | 3.0 | 6.0 |
| Ethylhexyl | 5 | | 4 | 4 |
| methoxycinnamate | | | | |
| Uvinul T150 | | 2 | | 1 |
| Eusolex T-AVO ® | 2 | | | |
| Uvasorb Heb | 1 | | 1.0 | |
| Butylmethoxydibenzoyl- | 2.5 | | 2 | 2 |
| methane | | | | |
| Tinsorb S | 2 | 1.5 | 1.0 | |
| Vitamin E acetate | 0.2 | | 0.3 | 0.3 |
| Na₂H₂EDTA | | | | |
| Perfume, preservatives | | | | |
| Dyes, etc. | | | | |
| Sodium hydroxide | | q-s. | q.s. | |
| Triethanolamine | q.s. | | | q.s. |
| Water | to 100.0 | to 100.0 | to 100.0 | to 100.0 |

1. A method for photostabilizing a photoactive, photosensitive, or photostable compound comprising combining said compound with a compound of the formula I 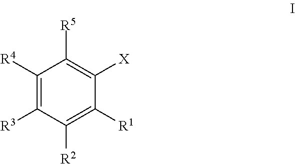
where X stands for 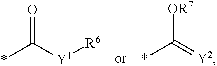
where the radical Y¹ stands for a single bond or the radicals Y¹ and Y² stand for a CR⁸R⁹ group,
where the radicals R¹ to R⁵ each, independently of one another, stand for H, Hal, CN, NH₂, OH, C(═O)R¹⁰, R¹¹ or OR¹¹,
where the radicals R⁶, R⁸ and R⁹ each, independently of one another, stand for H or R¹¹,
where the radical R⁷ stands for H, Si(R¹²)₃ or R¹¹,
where the radical R¹⁰ stands for OH, Hal, NH₂ or OR¹¹,
where the radicals R¹¹ each, independently of one another, stand for a straight-chain or branched C₁- to C₂₀-alkyl group or a straight-chain or branched C₂- to C₂₀-alkenyl group having at least one double bond, which may contain at least one OH bonded to a primary or secondary C atom, or
for a C₃- to C₈-cycloalkyl group or for a C₃- to C₈-cycloalkenyl group having at least one double bond,
where the radicals R¹² each, independently of one another, stand for a straight-chain or branched C₁- to C₈-alkyl group,
where Hal stands for F, Cl, Br or I,
and/or salts thereof as photostabiliser.

2. Method according to claim 1,
characterised in that Y¹ stands for a single bond and R⁶ stands for an H atom.

3. Method according to claim 1,
characterised in that the radicals Y¹ and Y² stand for a CR⁸R⁹ group.

4. Method according to claim 1, characterised in that the radicals R⁶ and R⁸ stand for H.

5. Method according to claim 1, characterised in that the radical R⁹ stands for H or R¹¹.

6. Method according to claim 1, characterised in that the radicals R¹¹ each, independently of one another, stand for a straight-chain or branched C₁- to C₈-alkyl group or a straight-chain or branched C₂- to C₈-alkenyl group having at least one double bond, which may contain at least one OH bonded to a primary or secondary C atom.

7. Method according to claim 1, characterised in that at least two of the radicals R¹ to R⁵ stand for an H atom.

8. Method according to claim 1, characterised in that the radical R³ stands for R¹¹ or OR¹¹.

9. Method according to claim 1, characterised in that the radicals R¹² in Si(R¹²)₃ each stand, independently of one another, for a straight-chain or branched C₁- to C₄-alkyl group.

10. Method according to claim 1, characterised in that a compound to be stabilised by at least one compound of the formula I has an energy level of a first triplet state, determined by a calibrated calculation method, in the range from 2.8 eV to 3.2 eV and/or in that an energy level of a first triplet state, determined by the calibrated calculation method, of the compound to be stabilised differs by a maximum of +/−5% from an energy level of the first triplet state, determined by the calibrated calculation method, of the compound of the formula I.

11. Method according to claim 1, characterised in that a first triplet state of an appearance form of the compound to be stabilised which tends towards photochemical decomposition is photostabilised.

12. Method according to claim 1, characterised in that an organic UV filter is photostabilised.

13. Method according to claim 1, characterised in that the compound of the formula I is used for photostabilisation in a cosmetic and/or dermatological and/or pharmaceutical composition and/or in foods and/or in food supplements and/or in packaging materials and/or in household products.

14. Composition comprising at least one vehicle which is suitable for cosmetic, pharmaceutical, dermatological compositions, foods, food supplements, household products or packaging materials and at least one compound of the formula I.
